SEQUENCE LISTING (31 sequences)

SEQ ID NO: 1 - DNA, 18 bp, artificial sequence
  primer targeting region P1 (first region), sense primer
accgccgccccccgtgag

SEQ ID NO: 2 - DNA, 23 bp, artificial sequence
  primer targeting region P1 (first region), antisense primer
ccacccgttgcccggacttcacc

SEQ ID NO: 3 - DNA, 26 bp, artificial sequence
  primer targeting region P2 (second region), sense primer
tactgtagcccctcttcaacacactc

SEQ ID NO: 4 - DNA, 19 bp, artificial sequence
  primer targeting region P2 (second region), antisense primer
tgcctgccagggcctctat

SEQ ID NO: 5 - DNA, 16 bp, artificial sequence
  primer targeting exon 2 of SLC6A8, sense primer
cctcttcaacacactc

SEQ ID NO: 6 - DNA, 18 bp, artificial sequence
  primer targeting exon 2 of SLC6A8, antisense primer
gatagtaacagtaggacc

SEQ ID NO: 7 - DNA, 15 bp, artificial sequence
  primer targeting exon 3 of SLC6A8, sense primer
cacacaagggggagc

SEQ ID NO: 8 - DNA, 17 bp, artificial sequence
  primer targeting exon 3 of SLC6A8, antisense primer
gagtgaccaggacagtg

SEQ ID NO: 9 - DNA, 18 bp, artificial sequence
  primer targeting exon 4 of SLC6A8, sense primer
tgggctgtgggagagaag

SEQ ID NO: 10 - DNA, 33 bp, artificial sequence
  primer targeting exon 4 of SLC6A8, antisense primer
cttccttagggctagactctgtctggtcacctc

SEQ ID NO: 11 - DNA, 16 bp, artificial sequence
  primer targeting P3 region (third region) of SLC6A8, sense primer
gccagcctcgctcctg

SEQ ID NO: 12 - DNA, 25 bp, artificial sequence
  primer targeting P3 region (third region) of SLC6A8, antisense primer
gggttaaagctgctacacagaagac

SEQ ID NO: 13 - DNA, 18 bp, artificial sequence
  primer targeting exons 5-6 of SLC6A8, sense primer
gcttccccactgcacttg

SEQ ID NO: 14 - DNA, 20 bp, artificial sequence
  primer targeting exons 5-6 of SLC6A8, antisense primer
agtggatgggttaggctgct

SEQ ID NO: 15 - DNA, 18 bp, artificial sequence
  primer targeting exons 5-7 of SLC6A8, sense primer
acctctgaacatacctgc

SEQ ID NO: 16 - DNA, 18 bp, artificial sequence
  primer targeting exons 5-7 of SLC6A8, antisense primer
acaaatcccaggacctgc

SEQ ID NO: 17 - DNA, 19 bp, artificial sequence
  primer targeting exon 5 of SLC6A8, antisense primer
tccccgcatctatccacac

SEQ ID NO: 18 - DNA, 18 bp, artificial sequence
  primer targeting exons 6-7 of SLC6A8, sense primer
gtgtggatagatgcgggg

SEQ ID NO: 19 - DNA, 18 bp, artificial sequence
  primer targeting exons 6-7 of SLC6A8, antisense primer
cgtttgaatcctcgtcac

SEQ ID NO: 20 - DNA, 15 bp, artificial sequence
  primer targeting exons 8-10 of SLC6A8, sense primer
gccgcacgacccagg

SEQ ID NO: 21 - DNA, 18 bp, artificial sequence
  primer targeting exons 8-10 of SLC6A8, antisense primer
atacaggcaatgtcgtcc

SEQ ID NO: 22 - DNA, 18 bp, artificial sequence
  primer targeting exon 8 of SLC6A8, antisense primer
ccctttccttgcctgcag

SEQ ID NO: 23 - DNA, 18 bp, artificial sequence
  primer targeting exons 8-9 of SLC6A8, antisense primer
gccaccagaggtcacagg

SEQ ID NO: 24 - DNA, 18 bp, artificial sequence
  primer targeting exons 9-10 of SLC6A8, sense primer
cggagggagggctgcagg

SEQ ID NO: 25 - DNA, 18 bp, artificial sequence
  primer targeting exon 11 of SLC6A8, sense primer
tacggtaggtcatggctg

SEQ ID NO: 26 - DNA, 18 bp, artificial sequence
  primer targeting exon 11 of SLC6A8, antisense primer
gatgaagatgccctacgg

SEQ ID NO: 27 - DNA, 19 bp, artificial sequence
  primer targeting exon 12 of SLC6A8, sense primer
cgctggtctgcatggtaag

SEQ ID NO: 28 - DNA, 20 bp, artificial sequence
  primer targeting exon 12 of SLC6A8, antisense primer
acttcctctgcctctctccg

SEQ ID NO: 29 - DNA, 20 bp, artificial sequence
  primer targeting exon 13 of SLC6A8, sense primer
cggagagaggcagaggaagt

SEQ ID NO: 30 - DNA, 19 bp, artificial sequence
  primer targeting exon 13 of SLC6A8, antisense primer
gtcagggaaaggcaggccc

SEQ ID NO: 31 - DNA, 15289 bp, Homo sapiens
  misc_feature: SCL6A8 gene, NCBI accession no. NG_012016
cacctggttaattttttgtatttttagtagagacggggcttcaccatgttagcctggatggtcttgatctcctgacctcgtgatccacccatgttggcctcccaaagtgttgagattacaggcatgagccaccgtgcccagccctgaaggctaggttttttttttgagacagagtctcactctgttgcccaggctggagtgcagtggcacaatcttggctcactgcaacctccacctcccgggttcaagcgatttccggctaacttttgtatttttagtagagacaggggtttcaccatgttggccaggctgctcttgaactcctgacctcaagtgacccacccgcctgggcctcccaaagtgctaggattacaggtgtgagccaccgcacccagcccgaaagctaggattttttaaagatagtttggcggacagggggctagggaatgggtgctgctgactggttgggtgggggatgattcttgtgtgctgagctgagtctgcttttaggtagggccacaggaccttgagtcataggtctatgtggtccgggtggagccatctggtagtgagaaatgcaaaaacctgcaaagacgtctcaaaaagccaaccttaggttctacaatagtgacattatctacaggagtaattggagaagttacaaatctcttgagctctgaacaatggctggtcatcatgaatgcttccatgttagcagaattcaggcccctctcatcctcctcacctgatggcctttcattacttttacaaaggcggtttcatcttgggaaggtctgttatcatttaaactataaacgaaatttctcccaaagttagcttggcccatgcccaggaaagaccaaaaacagtttggagggtaaatgcagacagggttggttagatcagctctctcactggcagaattttgttactgttacagtttttgcaaggcagctttagggtgatgggtctgcacggaatatatgcatgtaacagaaccgcgcttgtaccctctcaatctataaaacaaataaaaccagcctaataaaagtttacataaaatgtaaaaaacaaagcaaagcctcctttctgcgggtctgtgtaaacgagcacagctggtgggaagggcgcgggtggggggttctgctgccccccatccctgccctgctgcaggccctcgcccccagccccattctttctgttctccgcttggctgcagccgcacgtcggccccctccccaggagctggaagtacaaagcccttccaggtggactctggctccccctttgttcccagcttattctaattccaaagctcattgtgcccggctcgccttcagaagaggaggcgcccccatcctgtctccagctgcccatcctcccaggataaccagtcaccccagggcccggtggcccctcacccagcccctccccggtccgcagctgccctaggcttgagtgggcgctggctccaattctcaggcctcccccaacaaacaggagcattccggctagcccccctcccctgccctccccccagctccccttctcctcccctctccctcctcctcagctcctactccaaccccccagccccagctggggcctgaaaggctgcccactccctgggacacggtaaggggagggtgcagctctccccccgcccctccccggtcgcctctgccccagagaacagtttgcttctcacccagaagccaccataggagctctgggctgggcacaggtcgcagggcacccccaccccctcctgcacatgctcggaaccccccttcagtgagtagaacacaagggcctggcaagacaggcggaggcttggaaagggctggcgggggacagctacccggccctcagctgggggcctggagagcccaccctgcccctccccagcagctgctgcccccgggccgagcctgacgcgccttgacaaagcccgagaacgctttgaagccttcggacgtgggagaggacccagccagggatggaaatcgctttgcctttgttcccccaactgctcagcagctcgtgggagcagcctcagaagaccctgtcactggccgcccgggtcagagcctgtgacagagaccagagctccggccggagctccccgccggaaactccagccttaccagcctgaacttcatgcactgctcaaagaggcccagctcgccctggctgggcaggggcggctcccagggtggccgacgtggggagggttcctgaacctccctcaggagcctcctgggacagagtcctggaggtcagcagagaaagggaagcccaggctgtgggccccgcgtggagggaaaactcagggggaacgccccgaggctgggagggcacagccccatcacactccatctcgtagtcttggagaaaaatttagttctacctccagggcacagaaaagcccaaagaagggatgagataaaggaggggctggtcagatttgtgcttcaggagggtccctctggctgctcagagaatgcagtttggggtctggcccaggccgtgaggaggagttcacagtcactatggggtccagaccagctgggggcgggaggcagggcctggcccaggggctggtagtgggtgcaccccaggctgggctggcaaagggcggggaagaaggtgggtgtgggaaggggtggctgcctggagaagagcccagttccaggaggtcgtgggagtcaggtatgggggcccactggacctcccctcagatgtgggggctcccactccgagggtccatggagcagtggcagccattctggacagccccccacccttcacttctgtctccagtaccctccagcctggccacctcgccctctgcctcggcctcctctgatctcacaaaggcagcagcagtcaggggtggcgactccctcatgatcctgtccctggcctcggaacccaccggaggagtaaccgcagcagtggtcacttccccaaagtgccatcagctctcctacatccttctcgagcctctgctttctctcccttccacccccacccctttgacaagcaacatctgaaagtctcccctgcccgcggctcccagagctgtctggccgtggcctgcactctccctcaaagcggccctcccccaggtcaccgtcctcccttcgatgacatcacgcgcccaccccgcactcctgctgggctgaggccctccgagcctacttcaccgggtcctcttctttctctcaaaggcaatggggtttgcctgtggtccagctgggttggccctcttccctctgtcctgggtccttgagtggccccgcttctttaggccatctatgagtcacttctctaatgcccctgtctccagaccagcttcagtcaaaggctgggccagagaagaccctagtgagaaacttctgatgagcagtgtgaccttgccacctcaggggtacccacccaccacccctggtctaagcacaggtgacaccgcctgtctcccccaaccacacacaccccttgaggctcctcctccaagcctgggtggggacactgtccctccctcacccagcaagctcaatctggcttgggccggaactgcttttcttcctaaagctggacggatggccgcgggcttagcttaacgggatgagccatctggggactgcagtgtccacgatcagatcagggagcttgaagctgaggggggcacactttacctcccaggccaggacaatgaccacttccttccccaccccacccccaggctactcttagccctagaaaattctaaacaagctgctcagctggcggcggagaggcagcccaacaagctggctcttgctagggaggcctggggggtcctggggagaggaacacggggtgggtggggggcgggcagccaggacctcaggcctgaggcctttggggaagggtctgtgcacctgccaggcaccagggggcagccttgccttgttcccgctccagtcccctcaagtccgaagcccctacccactctcacgccaggcaggggtgggggccgccggggtcatttacccgggccccttctctgccttgatgacaaagtcgaggcttgctcatcagccaggcaggctcccctctgcccactgtggagacacagaggcctgtcacctgaagagctggtcccggcctccagcttccagggtagccgggaagctgtagcccccagtgggcagcggtggagagagctcaaggaaggagggagcaccgggaggagacggctgcagcctgccaggagcggggagaaagggagagaaggggaggcggagggctgagggggcccgggggacgtcttcccagggctgggaggggccggccgggaagcctgggctgcactaggagccggcgaccctggggcgaggggcggcccggagccctgcgggaggagctggcggccgccccaggtagcaaccatcctgcctcccgctggagcggcgtctcctccccgggaggagggcagggaggaggtgggcggagtgtgacgaggagggcgggagggagggatgcgggagggggagggggaggggggccggccggccgtgggggtggggcgatagtgacatcaccccggagtcggtttttaagcggcggccggccggggacggggaagagagggatagtcggagcgaggtggcgagtcgctgagcccgccgcggccccgagagcggctgcagccgccgccgccgggaaggagagggcgaggcgcgcccgagccgccgccgccgccgccaccgccgccgccgccaccaccgccaccggagtcgcgggccagccgggcagcctccgcgggccccggccggggcggggggcgcgggccacaggcccctgctccggccgccgcttgcagaccgcgggcgccgatgtcgcccgcgccccgctaggctgagcctcgggtcgggcgaggagccgccgcagccgccgccgcccgagccgcgggcaggagcctcgggagccgccgccgccgccgccgccgcccggccgggccccgccgccgcccgcgcgcccccgggcccccgacacacatgagattcttcaggctcactttcaagtgcttcgtggactgcttctgactgcgccgcccgcgccccgcaccccgccgcccgcccgccgccccgtcccccggcccggccgccccccggcccccggccggcccgcgccctcggggccctccccggtgccgccggtgccccccgcctgaccgccgccccccgtgaggcgccgcgaccccggcccggccgtgcggcccgccgaggccatggcgaagaagagcgccgagaacggcatctatagcgtgtccggcgacgagaagaagggccccctcatcgcgcccgggcccgacggggccccggccaagggcgacggccccgtgggcctggggacacccggcggccgcctggccgtgccgccgcgcgagacctggacgcgccagatggacttcatcatgtcgtgcgtgggcttcgccgtgggcttgggcaacgtgtggcgcttcccctacctgtgctacaagaacggcggaggtgagttcccccgcccgccgcggcctcctcccccagcaggccgccggcccccgcccgacccccggagccgccgcggaggggtgaagtccgggcaacgggtggcccccgggcacgcgggggtcggggccgcccctcgtccgccgctgccgctcggtggccgggccgggcgcctccacccccctcgcagtcatgtgcctggcatggtggggggagggggccggcgatgcccgcgaggctgccccccagactcccgggctgggaggagcgattggccgccgaggtgggaaagcaggcctgcgccttggggtctccgcgaggtaaggagccctggctgcccccacgggtcgggcacacaagcggcacattgtgtgggccccccacgtgtgcacacacacgaacacacacacacacaatgggccactctgtccctccccctgccctcccctcccctcgcggccctcccgcccctcccctctggcccgggcctggaacactgggtgcccgagccaggcttgggaagcctgcggcctggcccgcctggcgccgccactggacacactgcatgcacgtcccatgcccgcccgcccgcccgcccgcccgggcccagcttagcaacagcgatgggcacgcgtgtgtcctgtgactacaaaacagcactggggttgctggaagccgaagtgacccggtgatgggtgggaaacagaggtccagagcaaaggcctttgcccaaggtcaggagaaggatgctgggacctggagtcaggcaagttgcagccaagctcagcctctgagtagtggagcgagcccagccagggcaagggtaggaggcccagagaggagaagggggtagtggcacccagctctccctgcccttctgccacccccaccccagcctgctggcctcaggagataggcctgtgtcacgccctgcctatctcctgcagagcctgactccctggccttgctaaggccggcctggcccctcttccgcacctgtatccctctgtccttgcacatcgccatcccaccagcaggggactgtgacccacccaccctctgccttagacctcacacttgcaggcaagcgtccaagggcaggacagtcgcgctccctgcctttggatgagccccccaggcctgatcacccagccttggcacacatgcacacatgcacgtgccctcactgtgctgcctgaaacagggaattgcagcactagggacagcccgcgtgtctgagcgtgtgtgtcctccatggccatcgccccaagtgaccgtgggggtggaagccctgggggcctagggcccctctgccacccagggaatagggctccaatggctcaggggctactgtagcccctcttcaacacactcaacccaccccctcaagactccacctggggcctgagtcagtggccacccctacactgactcacccagtcggaagttgtgatggggcctttggagtctgggctggcccgctgggcctgggcagcctggctgggggccaccctgagtccacgctgtgcctccacccccaggtgtgttccttattccctacgtcctgatcgccctggttggaggaatccccattttcttcttagagatctcgctgggccagttcatgaaggccggcagcatcaatgtctggaacatctgtcccctgttcaaaggtgagcagcccttggccagcctcagggactgcccccttctcccagctggctcccacttgagaaatcttttcctgtcgtgagcaccaggcctggggccacgtgatggcgtcccagtctcgaggggggagcctggaggagatgttcaggccgcacagcgaacttggggaagcggggactagagggggcataggcagctccacaaggcaaggacaggccaggcatagccgggctggggacgggacctgcccagcagcacccttggctctctaggtaggtcctactgttactatccccaaggacgctggggcacagacaggtggagcgacgtactgaggttgcccactgcaggggcgactgtctccaacactacctcaggcgactagaaaccccccccccccaccaccaccatcaacaccagctgctgaggactggaggctactgggtggccaggcagaggcttggacctcctggaaccgccatggtggcagtgggacccacagaaggggccaggtgtatgaggctggagactccacagcacttggtcagatggggacaggaggagaggggctcgctctgccttgggtctagggggcggctggaggagaggagacaggctggggagtcagcgcagtgttggggctcacacaagggggagcccaggggagtcaggagcaccacaaacaaggctccaggaggacagatggtgggagcacggccagcctgggtggggacataaaggggtggcagggggaggtggccagggaagaatctacatggcaaggacttcccggccccaggcctgggctacgcctccatggtgatcgtcttctactgcaacacctactacatcatggtgctggcctggggcttctattacctggtcaagtcctttaccaccacgctgccctgggccacatgtggccacacctggaacactcccgactgcgtggagatcttccgccatgaagactgtgccaatgccagcctggccaacctcacctgtgaccagcttgctgaccgccggtcccctgtcatcgagttctgggagtgagtccggcacctctgggccaagcccatcccatcccccaggtctccctcatgttgcccggctccaggggagtggccctgagggggcaccagggtgttgcctggcagtccatcctggaccctgcctgcccttgcctgtcctcggagagtcctggggccagcctcgctcctgggttcggcagccgatcactgtcctggtcactcccccctgatgggggagctggggctgcatgtgaggtgggatgggagtggcctcccaatggccaggggatcgtgggctccaggcccagcccaattggacaagagggacccgctgaaccctgggctgtgggagagaagggagccacaactcctgggggtggaccctgtggctccatcctctgctggcacaggcctcatgggacctccctccctcccctaggaacaaagtcttgaggctgtctgggggactggaggtgccaggggccctcaactgggaggtgaccctttgtctgctggcctgctgggtgctggtctacttctgtgtctggaagggggtcaaatccacgggaaaggtaccactagaggcatgcagcggggagggtggctcagccctgggagccggatgtctgtgccaggcacacctgtggcaacgggaggtgaccagacagagtctagccctaaggaagggggaggtactgaaagccaagcaatgctccccaccctgcaaatccagggcccagcagcctttgctcctggggatagaggccctggcaggcactgtcccttccctgtgcccatcacccccactggtgccctcctgccagtctctgactcttgtgacagtctggtggacctggtctggccatctgttacctatcttgccttggggacccagagcagagtctggccacatcccttgggggctcctggtcaggctggggagtcacctgaacaaagaagacagtgtctagagctgtgggacatggccagctccctgggggacaaggtccccagagcagcatgtgggaagagggggcagacagtgtggcagctgcatctcgcctgcctctgcctggcccagttccactctccacctgctcaacccccacctctctccagaagaggagggggacccgacccggatccaatatcccgctccctgcctgggcctcccacacctgcactgcccacacactcatacagctctcactccccacgtgctccacgcctcctgtccccactgaggagagctcccagaggctcgcctgctccccaccgacacgcgtccctgcagacaaacgaggcgcccagggagcttccccactgcacttggccagggctgccggggcgcagccttgcccctagcttcctctggcgggagccatggctcggaggacaatggggacctctgaacatacctgcccgcaagggggaccggaggcgctgggagtgggggtgtgagggaggtggtgccacagcctccgctgagcagcctggccccccagatcgtgtacttcactgctacattcccctacgtggtcctggtcgtgctgctggtgcgtggagtgctgctgcctggcgccctggatggcatcatttactatctcaagcctgactggtcaaagctggggtcccctcaggtgaggtggaggtggagaggctgcagcagggcgctgcgggggagccctgcaggcccctcatgcctgcgctctccggcccttctctaggtgtggatagatgcggggacccagattttcttttcttacgccattggcctgggggccctcacagccctgggcagctacaaccgcttcaacaacaactgctacaagtaagcaccgccgccctgccacccgtgccctgtcctgccctgccccgccctgcccagcagcctaacccatccactctggcccctccacccctcagggacgccatcatcctggctctcatcaacagtgggaccagcttctttgctggcttcgtggtcttctccatcctgggcttcatggctgcagagcagggcgtgcacatctccaaggtggcagagtcaggtagggccctacccccagccccgcctccagagcagcgagtgctacccagatgcatgatgtacaggaacatgcaatagaaatgctgaaaagtgacgaggattcaaacggaacttgtcagattgtgggcctgtgggggcaggtcctgggatttgtcaatgttgacagagaaaggacctcccagcccctgccgcacgacccagggttgacagcgcctctgaggcaggcgtgggcatgggcgcgagtgttgcaggcagggctcagggtgcgcacagggcaggacatcggctacaaggtctagagcctgcacctttcccacagggccgggcctggccttcatcgcctacccgcgggctgtcacgctgatgccagtggccccactctgggctgccctgttcttcttcatgctgttgctgcttggtctcgacagccaggtttgcatggggctctgggacagggagccaggaggggggcggagggagggctgcaggcaaggaaaggggtggagggcggtgcggggctcggcctgagctgccctggccacagtttgtaggtgtggagggcttcatcaccggcctcctcgacctcctcccggcctcctactacttccgtttccaaagggagatctctgtggccctctgttgtgccctctgctttgtcatcgatctctccatggtgactgatgtgagtggggtggggggtctgcctgtgacctctggtggccgtctgccatcctccctgactgggctctgtcccccagggcgggatgtacgtcttccagctgtttgactactactcggccagcggcaccaccctgctctggcaggccttttgggagtgcgtggtggtggcctgggtgtacggtaggtcatggctgagggctgggctgggggatggtggcggggaaggcaggtctccagcttggccctcccgcctcacctcgccgcaggagctgaccgcttcatggacgacattgcctgtatgatcgggtaccgaccttgcccctggatgaaatggtgctggtccttcttcaccccgctggtctgcatggtaagggctgggggaggtggggcagggcggggggcgaggcagggcggggtaggggccccattaaccgcagcattctggtccgtagggcatcttcatcttcaacgttgtgtactacgagccgctggtctacaacaacacctacgtgtacccgtggtggggtgaggccatgggctgggccttcgccctgtcctccatgctgtgcgtgccgctgcacctcctgggctgcctcctcagggccaagggcaccatggctgaggtaaggctcccgcccggcccgccctcccctcccctgctgtgaacattcaacccagcctgcttcctagccagggagtggccccgactagggtggcaggcagtgggaaccggagagaggcagaggaagtcaccgtggggacgagcaggtgaccctgggggcttcagcatgtcctcctctcctgcagcgctggcagcacctgacccagcccatctggggcctccaccacttggagtaccgagctcaggacgcagatgtcaggggcctgaccaccctgaccccagtgtccgagagcagcaaggtcgtcgtggtggagagtgtcatgtgacaactcagctcacatcaccagctcacctctggtagccatagcagcccctgcttcagccccaccgcacccctccagggggcctgcctttccctgacacttttggggtctgcctgggggaggaggggagaaagcaccatgagtgctcactaaaacaactttttccatttttaataaaacgccaaaaatatcacaacccaccaaaaatagatgcctctccccctccagccctagccgagctggtcctaggccccgcctagtgccccacccccacccacagtgctgcactcctcctgcccctgccacgcccaccccctgcccacctctccaggctctgctctgcagcacacccgtgggtgacccctcaccccagaagcagcagtggcagcttgggaaatgtgaggaagggaaggagggagagacgggagggaggagagagaggagaagggaggcaggggaggggcagcagaaccaaggcaaatatttcagctgggctatacccctctccccatccctgttatagaagcttagagagccagccagcaatggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaaaagccactgcagacatagcaataaaaacatgtcattttccaaagcaggctcctgcttccgcctctgctgctctaaggaaggggtcggggtacaggaggcagggggaacctcctccagctggagctgctgccgtgagcaaggctctgctctggaggcctctgcggccggcacccttctggggactgggaagggggcagggaaggcagcagcccaggggaaggccttgtccccctggagccgaggcagttggggagagcaggacgagagtgagctggagagcagccacacccgcggggaagggtgggcgtaaagccatgggtgctgaaattttcaaaatgttaccccaagaatttgtcactgaacaggtgccttgtgtcacttgggccaggctggtagcagcagaggggataactctgcatcagggatcaattttgaaggtggagccaataggggttgtgcatgaccaggatgcagggctcaaagaggagttaaggacaacagatttggcctgagcaagaggaaagatggagctgccaggtcctgcaatggggaggcaaggagagaatggtctggagtcagccttgggtgtgtcatgcaggaagtgtcatccaagtggagatgtctagttggcaggtggacacaggagttccagaaagtactggagatggaactgtgcaagttcttaccacatagagatgacactgaaagccctgagcctgagtgagctcacagggacgccgcaagccccggaacacaatgagaggggcagagcgaagacgtggcagtgataggggaggacgcctgagagttcctggtggggtcctgcaacctgagccagtgaggacccctcacaggtcagggaggagcagtggctggctccatctgtccagtgctgctgctggtgaaggacagtgacctgcaaatgctcactgagtctggcaagggtcacgggggcctggcgagggtggcttgcatgagcgggtgcgtgtgaaaggctgggtggtgtgcgactgagaaaaggagtggcggcagcgcagtgtcatctgcagacgaagggagagacaacaacgtagttcacccagacaaggaaatatgagccagcctggaaagggaaggcattccaacacacgacacaacatggctgaccctggagggcatttctgtgaaatgagccatcataaagggatacttgctatagggttctgctcctgtgagagagacagggccttacatgagaggagggagatccacagagacagagggcaagggtgggtgccaggggctggggacagggtggggagtgttgagtggggacagagtgtcagtttgagaaaataaattctagaggtggatggaagtggtggctgcgcaacactgtgactgcacttaatgccactgaattgcacatttaacgatggtgaaaatggctcattacatatacactgatgacactatatatatgtatgatatatatgcgttttaccatgagaagaggtggagaggaattggagacactgagtacagacaggtccttcaacgggcgggaccccgtgcacaagatgagcatgtggcaccccaccctcaaagggctgggcaccatggcagggcacagcaggcaatgcagtgggcggctcaggcaagcacagagagcatcagagattggagcctgtgaagggggagcaggtgacccctcagagcaaagtgacagcttgggctgctccctttgcgtcctgcccaggactgctatcgtgctatgggagaacccccagaggccctgctcctcagcaggcagcaccccctatggaggggctttacccctaaacttctggagccaggggagggacctggcttggaatacggccaaccaagagcctgggtgagaaatacacggaccagacagggagcagagaaaggagtggcagtgcagtcccaccctagctcagccaggggctctggagcctgtcctgcagtccctggcccccatctcttcagcaaccgctgtttccagttttctttttctccctgagaagcctgtcctct

(19) United States
(12) Patent Application Publication
(10) Pub. No.: US 2014/0377757 A1
(43) Pub. Date: Dec. 25, 2014

(54) COMPOSITIONS AND METHODS FOR SCREENING FOR CREATINE TRANSPORTER DEFICIENCY

(71) Applicants: Cincinnati Children's Hospital Medical Center, Center for Technology Commercializeation, Cincinnati, OH (US); University of Cincinnati, Cincinnati, OH (US)

(72) Inventors: Joseph F. Clark, Cincinnati, OH (US); Gail Pyne-Geithman, Cincinnati, OH (US); Yuko Kurosawa, Kusatsu (JP); Kejian Zhang, Cincinnati, OH (US); Wenying Zhang, Blue Ash, OH (US); Mehdi Keddache, Ludlow, KY (US)

(73) Assignee: UNIVERSITY OF CINCINNATI, CINCINNATI, OH (US)

(21) Appl. No.: 14/122,274

(22) PCT Filed: May 25, 2012
(86) PCT No.: PCT/US12/39672

§ 371 Date: Aug. 1, 2014

Related U.S. Application Data
(60) Provisional application No. 61/490,192, filed on May 26, 2011.

(51) Int. Cl. C12Q 1/68

(52) U.S. Cl. CPC C12Q 1/6883; C12Q 2600/156
USPC 435/6.12

(57) ABSTRACT

Amplification primers, sequencing primers, kits for screening, and screening methods for identifying a SLC6A8 creatine transporter gene mutation are disclosed. The screening method includes treating a sample of DNA with polymerase chain reaction amplification primers for amplifying regions of the DNA having SLC6A8 to produce a first, second, and third amplification product, sequencing the first, second, and third amplification products with sequencing primer pairs to provide a DNA sequence of SLC6A8 in the sample, and comparing the DNA sequence of SLC6A8 with a reference DNA sequence of SLC6A8.

This application claims priority under 35 U.S.C. §119 to U.S. Provisional Application Ser. No. 61/490,192, filed May 26, 2011, the contents of which are hereby incorporated by reference in their entirety.

The present disclosure relates to primers, kits, and screening methods, and, more specifically, relates to amplification and sequencing primers, kits for screening, and screening methods for identifying a SLC6A8 creatine transporter gene mutation.

There has recently been an increase in the diagnosis of autism spectrum disorders in the United States and worldwide. This is somewhat due to better diagnostic tests, more public awareness, and the development of effective treatments for some of the diseases that present in this spectrum. Recent estimates suggest that as many as 60 per 10,000 children show symptoms of autism. X-linked mental retardation (hereinafter “XLMR”) defines a group of these disorders, where the problem lies within the X chromosome; there are a wide range of mutations in known genes, and more are being discovered worldwide every year. In Western countries, mental retardation affects 2-3% of the general population. For most of these cases, the cause has not yet been determined, but the prevalence of affected males over females (approximately 70% males) indicates a significant contribution of XLMR to this patient population. XLMR is thought to account for 5-12% of all mental retardation (hereinafter “MR”). Approximately 2.1% of XLMR can be traced to a mutation in the SLC6A8 gene, which encodes the creatine transporter protein.

Creatine transporter deficiency (e.g. “CTD”) is an X-linked inborn error of creatine metabolism first described in 2001 based on a group of patients at the Cincinnati Children's Hospital Medical Center. Clinical hallmarks of this disease include mental retardation, expressive dysphasia, partial epilepsy and autistic features. The severity of these symptoms may be diminished with the early implementation therapeutic strategies, making early diagnosis essential. The older the child is before diagnosis, the harder it is to address developmental delays with therapeutic strategies.

The SLC6A8 gene is a member of a solute carrier family that is responsible for neurotransmitter transport. The SLC6A8 gene, located on Xq28, spans approximately 8.4 kb and consists of 13 exons. The protein encoded is 635 amino acids, with a predicted mw of 70 kDa. The SLC6A8 gene is thought to be expressed in brain, skeletal and smooth muscle as well as kidney. There is another form of the transporter, thought to be expressed only in testes.

Numerous mutations in the SLC6A8 gene have been identified that affect creatine transport and result in creatine transporter deficiency. Thus, identification of mutations within the gene causing creatine transporter deficiency requires analysis of the entire DNA sequence of the coding region of the SLC6A8 gene. The SLC6A8 gene is highly homologous with genes encoding other members of the solutes carrier family as well as a hypothetical protein (e.g. XP_{—}002343512) making it difficult to selectively amplify and sequence. For example, the hypothetical protein shares about 95% homology with the creatine transporter. Due to this homology, identification of mutations in the SLC6A8 gene has typically required sequencing the gene in many smaller sections using primers directed to short non-homologous sections of the SLC6A8 gene. Sequencing the entire gene in this manner is currently time consuming, laborious, and inefficient. Thus, primers and methods are needed to increase the efficiency of, as well as to decrease the time for, analyzing the SLC6A8 gene for mutations to increase the availability of screening for this disorder.

Currently, when diagnosing a child presenting with the spectrum of symptoms seen in creatine transporter deficiency, the child is first checked for mitochondrial electron transport chain dysfunction. If nothing is found, a skin biopsy (or increasingly, a blood draw) is performed to gather sufficient tissue for functional and genetic testing. The amount of skin that is obtained is not sufficient for genetic, functional, and biochemical analyses, and requires a painful procedure on the subject. It would be highly desirable to be able to collect a sample in a much less traumatic manner, and to be able to collect enough tissue for genetic, functional, and biochemical analyses.

Once the tissue is obtained, it is sent to a certified diagnostic laboratory with CLIA approval for a genetic test. There are currently only 6 laborites worldwide performing DNA-based sequence analysis of the entire coding region of SLC6A8, only three of which are located in the United States, and none of which perform creatine transporter kinetic testing to determine level of creatine transporter dysfunction. This process is a time-consuming, and sometimes traumatic, process in which precious time is lost that could have been used for implementation of early intervention strategies, which has been shown to improve the outcome of subjects that receive an early diagnosis.

The annual number of diagnoses for this deficiency is growing beyond the capabilities of the few laboratories capable of performing the tests, and currently employed diagnostic methods are incapable of being efficiently scaled up to handle screening of large numbers of newborns. This need is particularly glaring since the American College of Medical Genetics decided to not recommend screening newborns for the disorder because of the lack of a validated screen. Thus, methods and compositions for screening large numbers of subjects for creatine transporter deficiencies are needed.

Even in labs with diagnostic capabilities there is no attempt to adjust therapeutic strategies based on diagnostic findings. Succinctly, there is currently no laboratory in the USA with the scientific and expertise capability and creatine transporter patient population capable of comprehensive creatine transporter deficiency screening and/or functional profile with subsequent adjustments made to the therapy based on diagnostic findings.

The present disclosure is based on the discovery of novel amplification primers which selectively amplify first, second, and third regions of the SLC6A8 gene in humans to form first, second, and third amplification products. The present disclosure is also based on the discovery of novel sequencing primer pairs which sequence the first, second, and third amplification products. Accordingly, in one embodiment, a screening method for identifying a SLC6A8 creatine transporter gene mutation in a subject is disclosed. The method includes: (a) treating, under amplification conditions, a sample of genomic DNA from a human with a plurality of polymerase chain reaction (PCR) amplification primer pairs for amplifying a plurality of regions of human genomic DNA comprising SLC6A8, said treating producing a first amplification product containing the first region of SLC6A8, a second amplification product containing the second region of SLC6A8, and a third amplification product containing the third region of SLC6A8; (b) sequencing the first amplification product with a first set of sequencing primer pairs, sequencing the second amplification product with a second set of sequencing primer pairs, and sequencing the third amplification product with a third set of sequencing primer pairs, wherein the sequences from the first, second, and third amplification products align to provide a DNA sequence of SLC6A8 in the sample; and (c) comparing the DNA sequence of SLC6A8 with a reference DNA sequence of SLC6A8, thereby identifying said mutation. The first pair of amplification primers amplifies a first region of SLC6A8. The second pair of amplification primers amplifies a second region of SLC6A8. The third pair of amplification primers amplifies a third region of SLC6A8.

In another embodiment, a set of amplification primer pairs for selectively amplifying a first region, a second region, and a third region of a SLC6A8 creatine transporter gene is disclosed. The set of amplification primers includes a first pair of amplification primers for selectively amplifying the first region of SLC6A8, the first pair having SEQ ID NO: 1 and SEQ ID NO: 2; a second pair of amplification primers for selectively amplifying the second region of SLC6A8, the second pair having SEQ ID NO: 3 and SEQ ID NO: 4; and a third pair of amplification primers for selectively amplifying the third region of SLC6A8, the third pair having SEQ ID NO: 11 and SEQ ID NO: 12.

In yet another embodiment, a set of sequencing primer pairs is disclosed. The set of sequencing primer pairs includes a first set of sequencing primer pairs having SEQ ID NO: 1 and SEQ ID NO: 2; a second set of sequencing primer pairs having SEQ ID NO: 5 and SEQ ID NO: 6, SEQ ID NO: 7 and SEQ ID NO: 8, and SEQ ID NO: 9 and SEQ ID NO: 10; and a third set of sequencing primer pairs having SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

In yet still another embodiment, a kit for screening a subject for a SLC6A8 creatine transporter gene mutation is disclosed. The kit includes a set of amplification primer pairs and a set of sequencing primer pairs. The amplification primer pairs include a first pair of amplification primers having SEQ ID NO: 1 and SEQ ID NO: 2 for amplifying a first region of SLC6A8 to produce a first amplification product; a second pair of amplification primers having SEQ ID NO: 3 and SEQ ID NO: 4 for amplifying a second region of SLC6A8 to produce a second amplification product; and a third pair of amplification primers having SEQ ID NO: 11 and SEQ ID NO: 12 for amplifying a third region of SLC6A8 to produce a third amplification product. The set of sequencing primer pairs is for sequencing the first, second, and third amplification products. The set of sequencing primer pairs includes a first set of sequencing primer pairs having SEQ ID NO: 1 and SEQ ID NO: 2; a second set of sequencing primer pairs having SEQ ID NO: 5 and SEQ ID NO: 6, SEQ ID NO: 7 and SEQ ID NO: 8, and SEQ ID NO: 9 and SEQ ID NO: 10; and a third set of sequencing primer pairs having SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

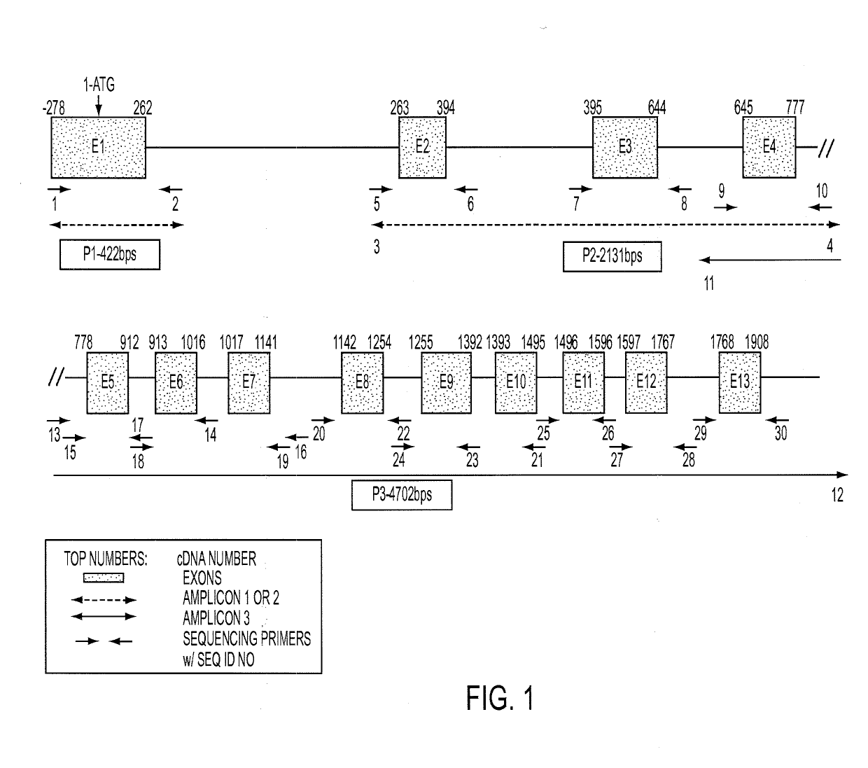

FIG. 1 is a graphical representation of the SLC6A8 gene amplification and sequencing strategy in accordance with embodiments of the present disclosure;

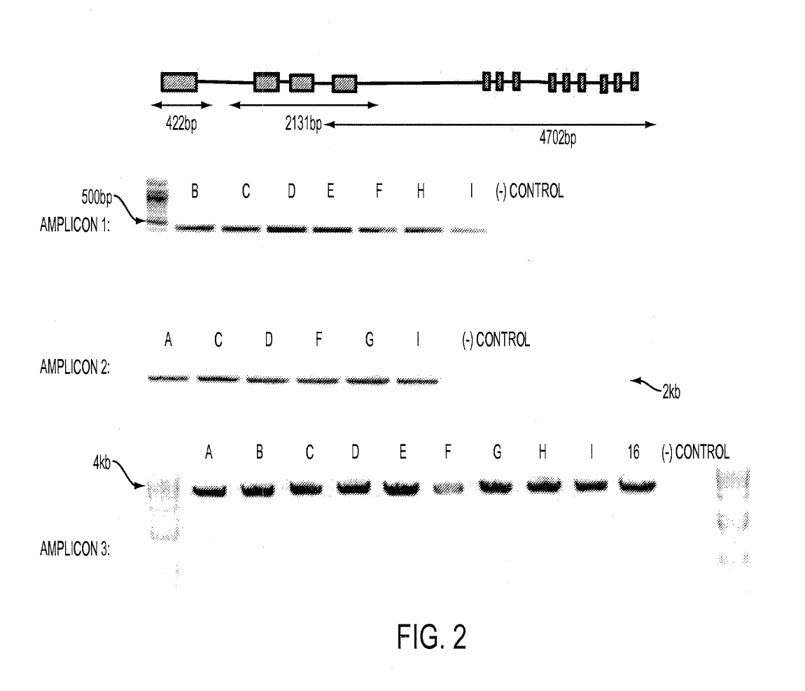

FIG. 2 includes a graphical representation of the SLC6A8 gene and representative photomicrographs of the amplification products of the fragments of the gene in accordance with embodiments of the present disclosure; and

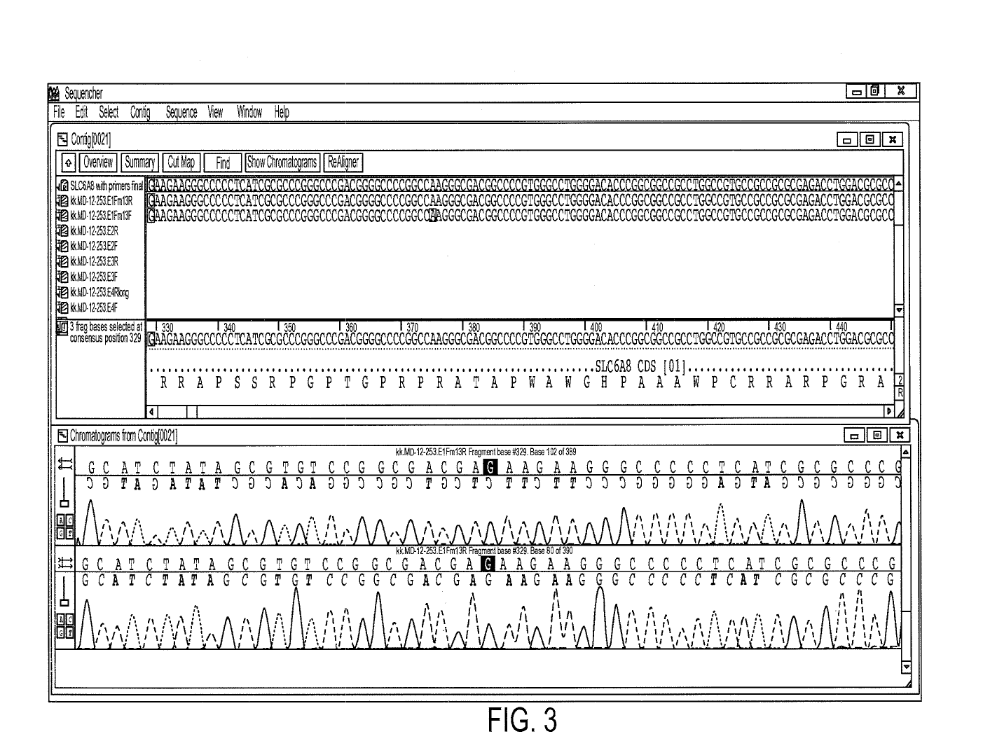

FIG. 3 is a screenshot of a sequenced portion of the SLC6A8 gene compared with a reference DNA sequence in accordance with embodiments of the present disclosure.

Skilled artisans appreciate that elements in the figures are illustrated for simplicity and clarity and are not necessarily drawn to scale. For example, the dimensions of some of the elements in the figures may be exaggerated relative to other elements, as well as conventional parts removed, to help to improve understanding of the various embodiments of the present invention.

The following terms are used in the present application:

As used herein, the terms “screening” and “screen” refer to a procedure by which the presence of a mutation in a SLC6A8 gene (i.e. SLC6A8) is determined. In one particular example, screening and screen refer to a procedure by which the presence of a mutation in SLC6A8 in human genomic DNA is determined.

As used herein, the term “mutation” refers to a variation in a DNA sequence or in a chromosome structure from that which is considered a normal or wild-type sequence or chromosome without defect. In the context of a DNA sequence, examples of mutations include point mutations, insertions, and deletions. Such mutations may have functional effects such as, for example, a decrease in function of a gene product, ablation of function in a gene product, and/or a new function in a gene product.

As used herein, the term “treating” refers to subjecting a sample to a chemical and/or biological process. In one particular example, treating refers to subjecting a sample of genomic DNA to a polymerase chain reaction.

As used herein, the term “amplification conditions” refers to conditions under which a region and/or regions of DNA may be amplified via cycles of denaturation, annealing, and DNA synthesis. In one particular example, the term amplification conditions refers to conditions under which: 1) a sample of double stranded DNA having SLC6A8 is denatured forming single stranded DNA; 2) a first, second, and/or third pair of amplification primers are annealed to the single stranded DNA and in particular to the first, second, and/or third region of SLC6A8; and 3) the primers are extended via DNA synthesis forming a new DNA strand complementary to the single stranded DNA. Such method performed under amplification conditions amplifies the first, second, and/or third regions of the SLC6A8 gene to produce a first, second, and/or third amplification product.

As used herein, the term “genomic DNA” refers to a population of DNA which holds the complete genetic component of a species. In one particular example, genomic DNA refers to a population of DNA which holds the complete genetic component of a human.

As used herein, the term “region” refers to a continuous sequence of nucleotides in DNA. With regard to SLC6A8, the term region refers to a continuous sequence of nucleotides in SLC6A8. In one particular example, SLC6A8 includes a first, second, and third region.

As used herein, the term “amplification product” refers to the region of DNA amplified via a polymerase chain reaction performed under amplification conditions. For example, in the context of amplifying a first, second, and/or third region of SLC6A8, the amplification product refers to the region of DNA amplified via repeated cycles of denaturation, annealing to a first second, and/or third pair of primers, and DNA synthesis.

As used herein, the term “sequencing” refers to a procedure for determining the order in which nucleotides appear in a DNA sequence. For example, with regard to SLC6A8, the term sequencing refers to a procedure for determining the order in which nucleotides appear in SLC6A8 and/or regions of SLC6A8. Examples of sequencing techniques include, but should not be limited to, chain terminator methods such as the Sanger method.

As used herein, the term “align” refers to the order in which nucleotides line up in a chain or chains of DNA. For example, in one particular embodiment, the term align refers to lining up the order of nucleotides in a first, second, and/or third amplification product amplified from a first, second and/or third region of SLC6A8. In one embodiment, the first, second, and/or third regions of SLC6A8 overlap, such that the nucleotide sequences of the first, second, and third amplification products overlap at the 5′ and/or 3′ ends. In this embodiment, the sequences of the first, second, and third amplification products can thus be aligned in order to provide a DNA sequence of the SLC6A8 gene.

As used herein, the term “comparing” refers to a procedure by which the order of nucleotides in a DNA sequence may be examined against the order of nucleotides in a reference DNA sequence to identify a mutation. Examples of tools employed to perform such procedure include, but should not be limited to, bioinformatic tools such as a basic local alignment search tool (i.e. BLAST), which is available from the National Institute of Health.

As used herein, the term “DNA sequence” refers to the ordering of nucleotides from which a molecule of DNA is composed.

As used herein, the term “reference DNA sequence” refers to a DNA sequence which does not contain a mutation and/or a DNA sequence which contains a known mutation or mutations.

As used herein, the term “evaluating” refers to a procedure by which function of a protein in a sample is determined. In one particular example, the term evaluating refers to a procedure by which the function of creatine transporter protein in a subject is determined.

As used herein, the term “creatine transporter assay” refers to a procedure by which the catalytic effect of creatine transporter protein is determined.

As used herein, the term “creatine transporter deficiency” refers to an X-linked inborn error of creatine metabolism. In one embodiment, creatine transporter deficiency is caused by a mutation in the SLC6A8 gene.

As used herein, the term “correlating” refers to a procedure by which a relationship between function of a creatine transporter protein and a mutation of the creatine transporter protein is determined.

As used herein, the term “selectively” refers to amplification primers and/or sequencing primers which anneal only to regions of SLC6A8 and/or to amplification products of SLC6A8. Stated another way, the term selectively refers to amplification and/or sequencing primers which do not substantially anneal to other known genes and/or amplification products.

As used herein, the term “high-throughput screening” refers to a screening method by which the presence of a mutation in a SLC6A8 gene (i.e. SLC6A8) in a relatively large number of samples is determined. In one particular example, the term high-throughput screening refers to a procedure by which the presence of a mutation in SLC6A8 in human genomic DNA is determined in a relatively large number of samples.

Embodiments of the present disclosure relate to screening methods for identifying a SLC6A8 creatine transporter gene mutation, a set of amplification primer pairs, a set of sequencing primer pairs, and a kit for screening a subject for SLC6A8 creatine transporter gene mutation. Reference will now be made in detail to embodiments of screening methods.

### I. Screening Methods

In one embodiment, a screening method for identifying a SLC6A8 creatine transporter gene mutation in a subject is provided. The screening method includes: (a) treating, under amplification conditions, a sample of genomic DNA from a human with a plurality of polymerase chain reaction (PCR) amplification primer pairs for amplifying a plurality of regions of human genomic DNA comprising SLC6A8, said treating producing a first amplification product containing the first region of SLC6A8, a second amplification product containing the second region of SLC6A8, and a third amplification product containing the third region of SLC6A8; (b) sequencing the first amplification product with a first set of sequencing primer pairs, sequencing the second amplification product with a second set of sequencing primer pairs, and sequencing the third amplification product with a third set of sequencing primer pairs, wherein the sequences from the first, second, and third amplification products align to provide a DNA sequence of SLC6A8 in the sample; and (c) comparing the DNA sequence of SLC6A8 with a reference DNA sequence of SLC6A8, thereby identifying said mutation. The first pair of amplification primers amplifies a first region of SLC6A8. The second pair of amplification primers amplifies a second region of SLC6A8. The third pair of amplification primers amplifies a third region of SLC6A8.

In one embodiment, the method includes treating, under amplification conditions, a sample of genomic DNA from a human with a plurality of polymerase chain reaction (i.e. PCR) amplification primer pairs for amplifying a plurality of regions of human genomic DNA having SLC6A8. The plurality of PCR amplification primer pairs includes a first pair of amplification primers which amplify a first region of SLC6A8, a second pair of amplification primers which amplify a second region of SLC6A8, and a third pair of amplification primers which amplify a third region of SLC6A8. The screening method amplifies human genomic DNA having SLC6A8 in three regions with a first, second, and third pair of amplification primers.

Referencing FIG. 1, with regard to the first, second, and third pair of amplification primers, the first pair of amplification primers may be employed to amplify the first region of SLC6A8 with short chain polymerase chain reaction. The first pair of amplification primers includes a sense primer (SEQ ID NO: 1) and an antisense primer (SEQ ID NO: 2), as set forth in Table I below.

TABLE I

| SEQ ID NO | DNA Sequeue |
|---|---|
| SEQ ID NO: 1 | ACCGCCGCCCCCCGTGAG |
| SEQ ID NO: 2 | CCACCCGTTGCCCGGACTTCACC |

The second pair of amplification primers may be employed to amplify the second region of SLC6A8 with long chain polymerase chain reaction. The second pair of amplification primers include a sense primer (SEQ ID NO: 3) and an antisense primer (SEQ ID NO: 4), as set forth in Table II below.

TABLE II

| SEQ ID NO | DNA Sequence |
|---|---|
| SEQ ID NO: 3 | TACTGTAGCCCCTCTTCAACACACTC |
| SEQ ID NO: 4 | TGCCTGCCAGGGCCTCTAT |

The third pair of amplification primers may be employed to amplify the third region of SLC6A8 with long chain polymerase chain reaction. The third pair of amplification primers include a sense primer (SEQ ID NO: 3) and an antisense primer (SEQ ID NO: 4), as set forth in Table III below.

TABLE III

| SEQ ID NO | DNA Sequence |
|---|---|
| SEQ ID NO: 11 | TACTGTAGCCCCTCTTCAACACACTC |
| SEQ ID NO: 12 | TGCCTGCCAGGGCCTCTAT |

In one embodiment, the first pair of amplification primers, the second pair of amplification primers, and the third pair of amplification primers selectively anneal to SLC6A8. Stated another way, the first pair of amplification primers, the second pair of amplification primers, and the third pair of amplification primers do not substantially anneal to other known genes under the amplification conditions.

Still referencing FIG. 1, with regard to first, second, and third region of SLC6A8, the first region of SLC6A8 includes exon 1. The second region of SLC6A8 includes exon 2, exon 3, and exon 4. The third region of SLC6A8 includes exon 4, exon 5, exon 6, exon 7, exon 8, exon 9, exon 10, exon 11, exon 12, and exon 13. The first region includes 422 base pairs, the second region includes 2131 base pairs, and the third region includes 4702 base pairs. The first region includes nucleotide positions of c.1-25 to c.262+25 of SLC6A8. The second region includes nucleotide positions c.263-25 to c.777+25 of SLC6A8. The third region includes nucleotide positions c.645-25 to c.1908+25 of SLC6A8. The abbreviation “c.” is used herein to refer to a coding sequence of SLC6A8. The first, second, and third region of SLC6A8 constitute all or substantially all of the coding region of SLC6A8.

In one embodiment, the first, second, and third pair of amplification primers amplify the first, second, and third regions of SLC6A8. Amplification conditions are such that amplification of the first, second, and third regions may be conducted via cycles of denaturing DNA, annealing primers to the DNA, and synthesizing DNA. More particularly, amplification conditions are conditions under which: 1) a sample of double stranded DNA having SLC6A8 may be denatured forming single stranded DNA; 2) a first, second, and/or third pair of amplification primers may be annealed to the single stranded DNA and in particular to the first, second, and/or third region of SLC6A8; and 3) the primers may be extended via DNA synthesis forming a new DNA strand complementary to the single stranded DNA. In one embodiment, the plurality of amplification primer pairs amplifies the plurality of regions under substantially similar amplification conditions.

With regard to denaturing the double stranded DNA having SLC6A8, the double stranded DNA is heated to a denaturing temperature such that the double stranded DNA will melt forming single stranded DNA. In one particular example, the denaturing temperature is from about 94° C. to about 98° C.

With regard to annealing the first, second, and third pair of amplification primers the single stranded DNA, and in particular, to the first, second, and third regions of SLC6A8, suitable annealing conditions are dependent upon a variety of parameters including: temperature, ionic strength, sequence length, complementarity, and the content of guanine:cytosine base pairs (hereinafter “G:C content”). With regard to temperature, lowering the temperature in the environment of complementary nucleic acid sequences promotes annealing. With regard to ionic strength, ionic strength or salt concentration may affect the melting temperature, wherein small cations may stabilize the formation of duplexes by negating the negative charge on the phosphodiester backbone of the DNA. Finally, with regard to G:C content, high G:C content and increased sequence length may stabilize duplex formation due to increased hydrogen bonding. Accordingly, a high G:C content and longer sequence lengths may impact the annealing conditions by elevating the melting temperature.

In one particular example, the annealing temperature for the first pair of primers and the first region of SLC6A8 is from about 60° C. to about 65° C., or about 62° C. In another particular example, the annealing temperature for the second pair of primers and the second region of SLC6A8 is from about 58° C. to about 68° C. In yet another example, the annealing temperature for the third pair of primers and the third region of SLC6A8 is from about 58° C. to about 68° C.

With regard to extending the primers via DNA synthesis to form a new DNA strand complementary to the single stranded DNA, in addition to the first, second, and third pair of amplification primers, the synthesis requires the sample of genomic DNA having SLC6A8, deoxynucleoside triphosphates, buffer solution, divalent cations, monovalent cations, and a DNA polymerase (such as Taq polymerase). In one particular example, the synthesis requires a sample of genomic DNA from a human having SLC6A8, deoxynucleoside triphosphates, and DNA polymerase.

Referencing FIG. 1 again, as previously discussed, treating the sample of genomic DNA having SLC6A8 under amplification conditions produces a first amplification product (i.e. P1/amplicon 1), a second amplification product (i.e. P2/amplicon 2), and a third amplification product (i.e. P3/amplicon 3). In one embodiment, the first amplification product, the second amplification product, and the third amplification product overlap at the 5′ and/or 3′ ends, such that aligning the sequences of the amplification products provides a continuous sequence of SLC6A8 or a portion thereof.

The method also includes sequencing the first amplification product, the second amplification product, and the third amplification product. The first, second, and third amplification products are respectively sequenced with a first, second, and third set of sequencing primers pairs. More particularly, the first amplification product may be sequenced with a first set of sequencing primer pairs. The first set of sequencing primer pairs includes a sense primer (SEQ ID NO: 1) and an antisense primer (SEQ ID NO: 2). The first set of sequencing primer pairs may sequence the entire DNA sequence of the first region, including any exons, introns, promoters, and enhancers. With specific regard to the first region of SLC6A8, the sequencing primers may sequence a portion of the DNA including exon 1.

The second amplification product may be sequenced with a second set of sequencing primer pairs. The second set of sequencing primer pairs includes the following pairs: a sense primer (SEQ ID NO: 5) and an antisense primer (SEQ ID NO: 6); a sense primer (SEQ ID NO: 7) and an antisense primer (SEQ ID NO: 8); and a sense primer (SEQ ID NO: 9) and an antisense primer (SEQ ID NO: 10), as set forth in Table IV below. The second set of sequencing primer pairs may sequence the entire DNA sequence of the second region, including any exons, introns, promoters, and enhancers. With specific regard to the second region of SLC6A8, the sequencing primers may sequence a portion of the DNA including exon 2, exon 3, and exon 4. More specifically, the sense primer (SEQ ID NO: 5) and the antisense primer (SEQ ID NO: 6) may sequence a portion of the DNA including exon 2, the sense primer (SEQ ID NO: 7) and the antisense primer (SEQ ID NO: 8) may sequence a portion of the DNA including exon 3; and the sense primer (SEQ ID NO: 9) and the antisense primer (SEQ ID NO: 10) may sequence a portion of the DNA including exon 4, as set forth in Table IV below.

TABLE IV

| | | Relevant |
|---|---|---|
| SEQ ID NO | DNA Sequence | Exon |
| SEQ ID NO: 5 | CCTCTTCAACACACTC | 2 |
| SEQ ID NO: 6 | GATAGTAACAGTAGGACC | 2 |
| SEQ ID NO: 7 | CACACAAGGGGGAGC | 3 |
| SEQ ID NO: 8 | GAGTGACCAGGACAGTG | 3 |
| SEQ ID NO: 9 | TGGGCTGTGGGAGAGAAG | 4 |
| SEQ ID NO: 10 | CTTCCTTAGGGCTAGACT | 4 |
| | CTGTCTGGTCACCTC | |

The third amplification product may be sequenced with a third set of sequencing primer pairs. The third set of sequencing primer pairs includes the following pairs: a sense primer (SEQ ID NO: 13) and an antisense primer (SEQ ID NO: 14); a sense primer (SEQ ID NO: 15) and an antisense primer (SEQ ID NO: 16); a sense primer (SEQ ID NO: 15) and an antisense primer (SEQ ID NO: 17), a sense primer (SEQ ID NO: 18) and an antisense primer (SEQ ID NO: 19); a sense primer (SEQ ID NO: 20) and an antisense primer (SEQ ID NO: 21); a sense primer (SEQ ID NO: 20) and an antisense primer (SEQ ID NO: 22); a sense primer (SEQ ID NO: 20) and an antisense primer (SEQ ID NO: 23); a sense primer (SEQ ID NO: 24) and an antisense primer (SEQ ID NO: 21); a sense primer (SEQ ID NO: 25) and an antisense primer (SEQ ID NO: 26); a sense primer (SEQ ID NO: 27) and an antisense primer (SEQ ID NO: 28); and a sense primer (SEQ ID NO: 29) and an antisense primer (SEQ ID NO: 30), as set forth in Table V below.

The third set of sequencing primer pairs may sequence the entire DNA sequence of the third region, including any exons, introns, promoters, and enhancers. With specific regard to the third region of SLC6A8, the sequencing primers may sequence a portion of the DNA including exon 5, exon 6, exon 7, exon 8, exon 9, exon 10, exon 11, exon 12, and exon 13. More specifically, the sense primer (SEQ ID NO: 13) may sequence a portion of the DNA including exons 5-6; the antisense primer (SEQ ID NO: 14) may sequence a portion of the DNA including exons 5-6; the sense primer (SEQ ID NO: 15) may sequence a portion of the DNA including exons 5 and 5-7; the antisense primer (SEQ ID NO: 16) may sequence a portion of the DNA including exons 5-7; the antisense primer (SEQ ID NO: 17) may sequence a portion of the DNA including exon 5; the sense primer (SEQ ID NO: 18) may sequence a portion of the DNA including exons 6-8; the antisense primer (SEQ ID NO: 19) may sequence a portion of the DNA including exons 6-7; the sense primer (SEQ ID NO: 20) may sequence a portion of the DNA including exons 8, 8-9, and 8-10; the antisense primer (SEQ ID NO: 21) may sequence a portion of the DNA including exons 8-9, 8-10, and 9-10; the antisense primer (SEQ ID NO: 22) may sequence a portion of the DNA including exon 8; the antisense primer (SEQ ID NO: 23) may sequence a portion of the DNA including exons 8-9; the sense primer (SEQ ID NO: 24) may sequence a portion of the DNA including exons 9-10; the sense primer (SEQ ID NO: 25) may sequence a portion of the DNA including exon 11; the antisense primer (SEQ ID NO: 26) may sequence a portion of the DNA including exon 11; the sense primer (SEQ ID NO: 27) may sequence a portion of the DNA including exon 12; the antisense primer (SEQ ID NO: 28) may sequence a portion of the DNA including exon 12; the sense primer (SEQ ID NO: 29) may sequence a portion of the DNA including exon 13; and the antisense primer (SEQ ID NO: 30) may sequence a portion of the DNA including exon 13; as set forth in Table V below.

TABLE V

| | | Relevant |
|---|---|---|
| SEQ ID NO | DNA Sequence | Exon(s) |
| SEQ ID NO: 13 | GCTTCCCCACTGCACTTG | 5-6 |
| SEQ ID NO: 14 | AGTGGATGGGTTAGGCTGCT | 5-6 |
| SEQ ID NO: 15 | ACCTCTGAACATACCTGC | 5 and 5-7 |
| SEQ ID NO: 16 | ACAAATCCCAGGACCTGC | 5-7 |
| SEQ ID NO: 17 | TCCCCGCATCTATCCACAC | 5 |
| SEQ ID NO: 18 | GTGTGGATAGATGCGGGG | 6-7 |
| SEQ ID NO: 19 | CGTTTGAATCCTCGTCAC | 6-7 |
| SEQ ID NO: 20 | GCCGCACGACCCAGG | 8, 8-9, |
| | | and 8-10 |
| SEQ ID NO: 21 | ATACAGGCAATGTCGTCC | 8-9, 8-10, |
| | | and 9-10 |
| SEQ ID NO: 22 | CCCTTTCCTTGCCTGCAG | 8 |
| SEQ ID NO: 23 | GCCACCAGAGGTCACAGG | 8-9 |
| SEQ ID NO: 24 | CGGAGGGAGGGCTGCAGG | 9-10 |
| SEQ ID NO: 25 | TACGGTAGGTCATGGCTG | 11 |
| SEQ ID NO: 26 | GATGAAGATGCCCTACGG | 11 |
| SEQ ID NO: 27 | CGCTGGTCTGCATGGTAAG | 12 |
| SEQ ID NO: 28 | ACTTCCTCTGCCTCTCTCCG | 12 |
| SEQ ID NO: 29 | CGGAGAGAGGCAGAGGAAGT | 13 |
| SEQ ID NO: 30 | GTCAGGGAAAGGCAGGCCC | 13 |

Sequencing may be performed according to methods known to one of ordinary skill in the art. In one particular example, sequencing of the first amplification product, the second amplification product, and the third amplification product may be performed via a chain terminator method, such as the Sanger method. The sequences of the first, second, and third amplification products align to provide a DNA sequence of SLC6A8. More particularly, by amplifying and sequencing the first, second, and third regions of SLC6A8, all or substantially all of the coding region of SLC6A8 may be sequenced, including all exons, introns, promoters, and enhancers. As a result, a mutation in any region of the coding region of SLC6A8 may be identified. As such, the screening methods discussed herein allow for the identification of novel mutations which were previously unknown.

One of ordinary skill in the art would understand that individual regions and/or exons may be sequenced by employing the first, second, and third set of sequencing primer pairs previously discussed. For example, as an alternative to sequencing each of the first, second, and third regions of SLC6A8, a single region or subset of regions may be sequenced employing the sequencing primer pairs listed in Table V. As a further example, as an alternative to sequencing each of exons 5-13 in the third fragment, a single exon or subset of exons may be sequenced employing the sequencing primer pairs listed in Table V. Such methods may be applicable wherein relevant mutations occur only in one region and/or subset of exons of SLC6A8.

In another embodiment, the screening method includes comparing the DNA sequence of SLC6A8 with a reference DNA sequence of SLC6A8 to identify a mutation. The reference DNA sequence may include a DNA sequence of the SLC6A8 gene, identified as accession number NG_{—}012016 (SEQ ID NO: 31). Such DNA sequence (SEQ ID NO: 31) corresponds to the normal, or wild-type form of SLC6A8, without defect. Additionally, such DNA sequence (SEQ ID NO: 31) includes both coding and non-coding regions. Alternatively, the reference DNA sequence may include a DNA sequence which does contain a mutation. Such DNA sequences may be found at http://www.ncbi.nlm.nih.gov/pubmed/20717164 and at https://portal.biobase-international.com/hgmd/pro/gene.php?gene=SLC6A8.

In another embodiment, the sample is obtained from the blood of a subject, such as by a simple blood draw. However, any biological sample containing genomic DNA from the subject could be employed in the methods described herein.

In one particular embodiment, amplifying the first, second, and third region of SLC6A8 and sequencing the first, second, and third amplification products may be performed separately. For example, amplifying the first region of SLC6A8 and sequencing the first amplification product may be performed in a first receptacle, amplifying the second region of SLC6A8 and sequencing the second amplification product may be performed in a second receptacle, and amplifying the third region of SLC6A8 and sequencing the third amplification may be performed in a third receptacle.

In another embodiment, the method further includes evaluating function of creatine transporter protein in the subject. Such evaluation may include procedures by which the catalytic effect and/or activity of creatine transporter protein is determined. For example, such evaluation may include determining creatine transporter kinetics and/or accumulation of creatine in cells. With regard to creatine transporter kinetics, such analysis may include determining Kₘ and/or Vₘₐₓ by procedures known to those of ordinary skill in the art. In one particular example, such procedures may include performing a creatine transporter assay on the biological sample from the subject.

The function of the creatine transporter protein may be evaluated in a biological sample from the subject. The biological sample may be obtained from any cell expressing a creatine transporter protein. In one particular embodiment, the biological sample may be from cells which are easily obtained via minimally invasive techniques. Examples of such cells include lymphocytes, peripheral lymphoblasts, blood cells, and/or buccal cells. Additionally, the biological sample may also include biopsied tissue samples which include fibroblasts. Cells may be cultured and/or used directly in assays previously discussed to functionally characterize the creatine transporter protein.

In one embodiment wherein the DNA sequence of SLC6A8 as determined by the methods discussed herein includes at least one mutation, the method may further include evaluating the function of the creatine transporter protein in the subject and correlating the function of the creatine transporter protein with the mutation. In certain embodiments, the correlated mutation of the gene and function of the protein are indicative of a diagnosis of creatine transporter deficiency. Such correlation may aid a clinician in developing a treatment regimen, such as which creatine and/or creatine analogs may provide optimum treatment outcomes. Such correlation may also aid a clinician in predicting the potential severity of a particular mutation. For example, some mutations may result in a partially functional transporter, while others result in ablation of the gene product completely. Correlating the catalytic effect and/or activity of the creatine transporter protein may give the clinician insights into how aggressively the deficiency should be treated.

In another embodiment, the methods discussed herein are suitable for high-throughput screening of multiple samples simultaneously and/or rapidly.

### II. Amplification Primer Pairs

Embodiments of the screening methods for identifying a SLC6A8 creatine transporter gene mutation have been described in detail. Further embodiments directed to amplification primer pairs will now be described.

In one embodiment, a set of amplification primer pairs for selectively amplifying a first region, a second region, and a third region of a SLC6A8 creatine transporter gene is disclosed. The set of amplification primer pairs includes a first pair of amplification primers for selectively amplifying the first region of SLC6A8, the first pair having SEQ ID NO: 1 and SEQ ID NO: 2. The set of amplification primer pairs also includes a second pair of amplification primers for selectively amplifying the second region of SLC6A8, the second pair having SEQ ID NO: 3 and SEQ ID NO: 4. The set of amplification primer pairs also includes a third pair of amplification primers for selectively amplifying the third region of SLC6A8, the third pair having SEQ ID NO: 11 and SEQ ID NO: 12.

The first region, second region, and the third region of the SLC6A8 creatine transporter gene are as discussed in an earlier section. Additionally, the amplification primer pairs may be employed as discussed in an earlier section.

### III. Sequencing Primer Pairs

Embodiments of the amplification primer pairs have been described in detail. Further embodiments directed to sequencing primer pairs will now be described.

In one embodiment, a set of sequencing primer pairs is disclosed. The set of sequencing primer pairs includes a first set of sequencing primer pairs having SEQ ID NO: 1 and SEQ ID NO: 2; a second set of sequencing primer pairs having SEQ ID NO: 5 and SEQ ID NO: 6, SEQ ID NO: 7 and SEQ ID NO: 8, and SEQ ID NO: 9 and SEQ ID NO: 10; and a third set of sequencing primer pairs having SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

The sequencing primer pairs may be employed as discussed in an earlier section.

### IV. Kit for Screening for SLC6A8 Creatine Transporter Gene Mutation

Embodiments of the sequencing primer pairs have been described in detail. Further embodiments directed to kits for screening for SLC6A8 creatine transporter gene mutations will now be described.

In one embodiment, a kit for screening a subject for SLC6A8 creatine transporter gene mutation is disclosed. The kit includes a set of amplification primer pairs and a set of sequencing primer pairs. The amplification primer pairs include a first pair of amplification primers having SEQ ID NO: 1 and SEQ ID NO: 2 for amplifying a first region of SLC6A8 to produce a first amplification product; a second pair of amplification primers having SEQ ID NO: 3 and SEQ ID NO: 4 for amplifying a second region of SLC6A8 to produce a second amplification product; and a third pair of amplification primers having SEQ ID NO: 11 and SEQ ID NO: 12 for amplifying a third region of SLC6A8 to produce a third amplification product. The set of sequencing primer pairs is for sequencing the first, second, and third amplification products. The set of sequencing primer pairs includes a first set of sequencing primer pairs having SEQ ID NO: 1 and SEQ ID NO: 2; a second set of sequencing primer pairs having SEQ ID NO: 5 and SEQ ID NO: 6, SEQ ID NO: 7 and SEQ ID NO: 8, and SEQ ID NO: 9 and SEQ ID NO: 10; and a third set of sequencing primer pairs having SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

The first region, second region, and the third region of the SLC6A8 creatine transporter gene are as discussed in an earlier section. Additionally, the amplification primer pairs and the sequencing primer pairs may be employed as discussed in an earlier section.

The kit may include instructions to provide guidance on the use of the amplification primer pairs and the set of sequencing primer pairs for sequencing a first, second, and third amplification product. For example, the kit may include instructions concerning the amplification conditions to employ with regard to the amplification primer pairs and may also include instructions concerning the ability of the sequencing primer pairs to sequence specific areas of SLC6A8, such as provided in Table V. It is understood that the instructions would convey the use of the amplification primer pairs and the set of sequencing primer pairs as discussed in an earlier section and/or as related to the screening methods previously discussed.

### EXAMPLES

The following non-limiting examples illustrate the compositions and methods of the present disclosure.

### Example 1

### Amplification, Purification, Confirmation, and Sequencing of the First, Second, and Third Regions of SLC6A8 Gene

Experimental Protocol.

Amplification, purification, confirmation, and sequencing of the first, second, and third regions of SLC6A8 were conducted. Amplification was conducted via polymerase chain reaction (hereinafter “PCR”), wherein a master mix containing water, 10× buffer, MgCl₂, dNTPs and Taq polymerase was prepared. For amplification of the first fragment, a master mix containing 2 μl 10×PCR Buffer; 1.2 μl 25 mM MgCl₂, 0.4 μl 10 mM dNTP, 0.5 μl FastStartTaq polymerase, 4 μl 5×GC Buffer, 1 μl a sense primer (SEQ ID NO: 1) and an antisense primer (SEQ ID NO: 2) (10 μM each) and 9.9 μl sterile water was prepared. For amplification of the second and third regions, a master mix containing 5.0 μl 10×LA PCR Buffer II (Mg²⁺ plus), 8 μl dNTP Mixture (2.5 mM each), 0.7 μl TaKaRaLA Taq 5 U/μl, 10 μl 5×GC Buffer, 4 μl primer mix (10 μM each), and 12.3 μl sterile water was prepared. The primer mix included a second and third pair of amplification primers, including a sense primer (SEQ ID NO: 3) and an antisense primer (SEQ ID NO: 4) and a sense primer (SEQ ID NO: 11) and an antisense primer (SEQ ID NO: 12).

Tubes were labeled with an identifier for each sample, and the appropriate master mix and sample DNA (100-250 ng) were aliquotted into each respectively labeled tube. The total reaction volume for the first region was 20 μl. The total reaction volume for the second region and the third region was 50 μl. Two sample tubes were used for the third region.

The sample tubes were sealed and placed in a thermal cycler for amplification. With regard to the first fragment, the thermal cycler was run with the following reaction conditions: 1) 94° C. for 3 minutes; 2) 94° C. for 30 seconds; 3) 62° C. for 45 seconds; 4) 72° C. for 40 seconds; 5) steps 2 to 4 repeated for 34 cycles; 6) 72° C. for 5 minutes; and 7) 12° C. until removed from thermal cycler for additional analysis. With regard to the second and the third regions, the following reaction conditions were employed in the thermal cycler: 1) 94° C. for 2 minutes; 2) 98° C. for 10 seconds; 3) 68° C. for 45 seconds and decrease by 1° C. every cycle; 4) 68° C. for 5 minutes; 5) steps 2 to 4 repeated for 10 cycles; 6) 98° C. for 10 seconds; 7) 58° C. for 45 seconds; 8) 68° C. for 5 minutes; 9) 68° C. for 13 minutes; and 10) 12° C. until removed from thermal cycler for additional analysis. The amplified products of the first, second, and third regions were stored at 4° C. until ready for sequencing.

Following amplification, the molecular weights of the amplified products of the first, second, and the third regions were determined to confirm that the products had molecular weights corresponding to the molecular weights of the first, second, and the third regions of SLC6A8. More specifically, the molecular weights of the amplified products of the first, second, and third regions were determined using an agarose gel.

Following confirmation, the amplified products of the first, second, and third regions were purified. More specifically, ExoSap was added to the PCR product, and sample tubes were sealed and placed in a thermal cycler. With regard to the amplified product of the first region, 2 μl of ExoSap was added to 20 μl of PCR product. With regard to the amplified products of the second and the third regions, 4 μl of ExoSap was added to 50 μl of PCR product. The thermal cycler was run with the following reaction conditions: 1) 37° C. for 30 minutes; 2) 80° C. for 15 minutes; and 3) held at 12° C. until removed from thermal cycler for additional analysis.

Following purification, the amplified products of the first, second, and third regions were sequenced. More specifically, the amplified products of the first, second, and third regions were sequenced by preparing a sequencing master mixture containing 11 μl water, 3 μl 5× sequencing buffer, and 3 μl purified PCR product. Additionally, 1 μl of the corresponding sequencing primer pairs was added into each sample tube.

More specifically, with regard to the first region, the sequencing primers included a sense primer (SEQ ID NO: 1) and an antisense primer (SEQ ID NO: 2). With regard to the second region, the sequencing primers included a sense primer (SEQ ID NO: 3) and an antisense primer (SEQ ID NO: 5) and an antisense primer (SEQ ID NO: 6); a sense primer (SEQ ID NO: 7) and an antisense primer (SEQ ID NO: 8); and a sense primer (SEQ ID NO: 9) and an antisense primer (SEQ ID NO: 10). With regard to the third region, the sequencing primers included a sense primer (SEQ ID NO: 13), an antisense primer (SEQ ID NO: 14), a sense primer (SEQ ID NO: 15), an antisense primer (SEQ ID NO: 16), an antisense primer (SEQ ID NO: 17), a sense primer (SEQ ID NO: 18), an antisense primer (SEQ ID NO: 19), a sense primer (SEQ ID NO: 20), an antisense primer (SEQ ID NO: 21), an antisense primer (SEQ ID NO: 22), an antisense primer (SEQ ID NO: 23), a sense primer (SEQ ID NO: 24), a sense primer (SEQ ID NO: 25), an antisense primer (SEQ ID NO: 26), a sense primer (SEQ ID NO: 27), an antisense primer (SEQ ID NO: 28, a sense primer (SEQ ID NO: 29), and an antisense primer (SEQ ID NO: 30)

The sequencing master mixture was denatured at 95° C. for 3 minutes, placed on ice for 2 minutes, and then 2 μl of the sequencing mixture was added to bring the total reaction volume to 20 μl. The sample tubes were sealed and placed in a thermal cycler and run with the following conditions: 1) 96° C. for 1 minute; 2) 96° C. for 10 seconds; 3) 50° C. for 5 seconds; 4) 60° C. for 75 seconds; 5) steps 2 to 4 repeated for 24 cycles; and 6) 4° C. until removed from thermal cycler. The sequenced regions were then analyzed using gel capillary electrophoresis, but other routine methods are also suitable for use.

Experimental Results.

As shown in FIG. 2, the amplified products of the first, second, and third fragments had molecular weights corresponding to the molecular weights (i.e. Mw) of the first, second, and third fragments of SLC6A8. More specifically, as shown in FIG. 2, the amplified product of the first region (i.e. Amplicon 1) had a molecular weight of about 422 base pairs (i.e. bp), the amplified product of the second region (i.e. Amplicon 2) had a Mw of about 2131 bp, and the amplified product of the third region (i.e. Amplicon 3) had a Mw of about 4702 bp.

As shown in FIG. 3, a portion of the SLC6A8 gene was sequenced. More particularly, FIG. 3 shows an example of bi-directional high quality sequence traces covering regions of SLC6A8.

It is noted that terms like “preferably,” “generally,” “commonly,” and “typically” are not utilized herein to limit the scope of the claims or to imply that certain features are critical, essential, or even important to the structure or function of the claims. Rather, these terms are merely intended to highlight alternative or additional features that may or may not be utilized in a particular embodiment of the present disclosure.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

For the purposes of describing and defining the present disclosure it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

1. A screening method for identifying a SLC6A8 creatine transporter gene mutation in a subject, the method comprising:
(a) treating, under amplification conditions, a sample of genomic DNA from a human with a plurality of polymerase chain reaction (PCR) amplification primer pairs for amplifying a plurality of regions of human genomic DNA comprising SLC6A8, wherein:
a first pair of amplification primers amplifies a first region of SLC6A8,
a second pair of amplification primers amplifies a second region of SLC6A8, and
a third pair of amplification primers amplifies a third region of SLC6A8,
said treating producing a first amplification product containing the first region of SLC6A8, a second amplification product containing the second region of SLC6A8, and a third amplification product containing the third region of SLC6A8,
(b) sequencing the first amplification product with a first set of sequencing primer pairs, sequencing the second amplification product with a second set of sequencing primer pairs, and sequencing the third amplification product with a third set of sequencing primer pairs, wherein the sequences from the first, second, and third amplification products align to provide a DNA sequence of SLC6A8 in the sample, and
(c) comparing the DNA sequence of SLC6A8 with a reference DNA sequence of SLC6A8, thereby identifying said mutation.

2. The method of claim 1, wherein the first pair of amplification primers comprises SEQ ID NO: 1 and SEQ ID NO: 2.

3. The method of claim 1, wherein the second pair of amplification primers comprises SEQ ID NO: 3 and SEQ ID NO: 4.

4. The method of claim 1, wherein the third pair of amplification primers comprises SEQ ID NO: 11 and SEQ ID NO: 12.

5. The method of claim 1, wherein the first region of SLC6A8 comprises exon 1, the second region of SCL6A8 comprises exons 2 to 4, and the third region of SCL6A8 comprises exons 4 to 13.

6. The method of claim 1, wherein the first region contains nucleotide positions 1-25 to c.262+25 of SLC6A8, the second region contains nucleotide positions c.263-25 to c.777+25 of SLC6A8, and the third region contains nucleotide positions c.645-25 to c.1908+25 of SLC6A8.

7. The method of claim 1, wherein the first set of sequencing primer pairs comprises SEQ ID NO: 1 and SEQ ID NO: 2.

8. The method of claim 1, wherein the second set of sequencing primer pairs comprises SEQ ID NO: 5 and SEQ ID NO: 6, SEQ ID NO: 7 and SEQ ID NO: 8, and SEQ ID NO: 9 and SEQ ID NO: 10.

9. The method of claim 1, wherein the third set of sequencing primer pairs comprises SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

10. The method of claim 1, wherein the sample is obtained from blood of the subject.

11. The method of claim 1, further comprising evaluating function of creatine transporter protein in the subject.

12. The method of claim 11, wherein the evaluating comprises determining creatine transporter kinetics comprising Kₘ and Vₘₐₓ.

13. The method of claim 11, wherein the evaluating comprises performing a creatine transporter assay on a biological sample from the subject.

14. The method of claim 1, wherein when the DNA sequence of SLC6A8 comprises at least one mutation indicative of a diagnosis of creatine transporter deficiency, the method further comprising:
evaluating function of creatine transporter protein in the subject; and
correlating the function of the creatine transporter protein with the at least one mutation indicative of the diagnosis of creatine transporter deficiency.

15. A set of amplification primer pairs for selectively amplifying a first region, a second region, and a third region of a SLC6A8 creatine transporter gene, the set comprising:
a first pair of amplification primers for selectively amplifying the first region of SLC6A8, the first pair comprising SEQ ID NO: 1 and SEQ ID NO: 2;
a second pair of amplification primers for selectively amplifying the second region of SLC6A8, the second pair comprising SEQ ID NO: 3 and SEQ ID NO: 4; and
a third pair of amplification primers for selectively amplifying the third region of SLC6A8, the third pair comprising SEQ ID NO: 11 and SEQ ID NO: 12.

16. A set of sequencing primer pairs comprising:
a first set of sequencing primer pairs comprising SEQ ID NO: 1 and SEQ ID NO: 2;
a second set of sequencing primer pairs comprising SEQ ID NO: 5 and SEQ ID NO: 6, SEQ ID NO: 7 and SEQ ID NO: 8, and SEQ ID NO: 9 and SEQ ID NO: 10; and
a third set of sequencing primer pairs comprising SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

17. A kit for screening a subject for a SLC6A8 creatine transporter gene mutation, the kit comprising:
a set of amplification primer pairs comprising:
a first pair of amplification primers comprising SEQ ID NO: 1 and SEQ ID NO: 2 for amplifying a first region of SLC6A8 to produce a first amplification product;
a second pair of amplification primers comprising SEQ ID NO: 3 and SEQ ID NO: 4 for amplifying a second region of SLC6A8 to produce a second amplification product; and
a third pair of amplification primers comprising SEQ ID NO: 11 and SEQ ID NO: 12 for amplifying a third region of SLC6A8 to produce a third amplification product; and
a set of sequencing primer pairs for sequencing the first, second, and third amplification products, wherein the set of sequencing primer pairs comprises:
a first set of sequencing primer pairs comprising SEQ ID NO: 1 and SEQ ID NO: 2;
a second set of sequencing primer pairs comprising SEQ ID NO: 5 and SEQ ID NO: 6, SEQ ID NO: 7 and SEQ ID NO: 8, and SEQ ID NO: 9 and SEQ ID NO: 10; and
a third set of sequencing primer pairs comprising SEQ ID NO: 13 and SEQ ID NO: 14, SEQ ID NO: 15 and SEQ ID NO: 16, SEQ ID NO: 15 and SEQ ID NO: 17, SEQ ID NO: 18 and SEQ ID NO: 19, SEQ ID NO: 20 and SEQ ID NO: 21, SEQ ID NO: 20 and SEQ ID NO: 22, SEQ ID NO: 20 and SEQ ID NO: 23, SEQ ID NO: 24 and SEQ ID NO: 21, SEQ ID NO: 25 and SEQ ID NO: 26, SEQ ID NO: 27 and SEQ ID NO: 28, and SEQ ID NO: 29 and SEQ ID NO: 30.

18. The method of claim 1, wherein the first, second, and third pairs of amplification primers do not substantially anneal to other known genes under said amplification conditions.

19. The method of claim 18, wherein the plurality of amplification primer pairs amplify the plurality of regions under substantially similar amplification conditions.

20. The method of claim 19, wherein the method is suitable for high-throughput screening of multiple samples simultaneously.
